(19) United States
(12) Patent Application Publication
(10) Pub. No.: US 2023/0024911 A1
(43) Pub. Date: Jan. 26, 2023

(54) DEVICE FOR MEASURING VOLUMES OF A LIQUID IN A CONTAINER BY MEASURING AN EMITTED HIGH-FREQUENCY RADIATION

(71) Applicant: FRESENIUS MEDICAL CARE DEUTSCHLAND GMBH, Bad Homburg (DE)

(72) Inventors: Felix WEGE, Aachen (DE); David HANNES, Leverkusen (DE); Robert LINDEMANN, Wiesbaden (DE)

(73) Assignee: FRESENIUS MEDICAL CARE DEUTSCHLAND GMBH, Bad Homburg (DE)

(21) Appl. No.: 17/785,056

(22) PCT Filed: Dec. 17, 2020
(86) PCT No.: PCT/EP2020/086647

§ 371 Date: Jun. 14, 2022

(30) Foreign Application Priority Data
Dec. 17, 2019 (DE) 10 2019 219 816.3

(51) Int. Cl. A61M 1/14

(52) U.S. Cl. CPC A61M 1/14; A61M 2205/33; A61M 2205/3389

(57) ABSTRACT

The invention relates to device (1) for measuring volumes of a liquid in a container (B) by means of measuring emitted high-frequency radiation, comprising control unit (C), a transmitter (TX), at least one first transmitting antenna (ANT_TX1) and at least one second transmitting antenna (ANT_TX2), at least one receiving antenna (ANT_RX1) and a receiver (RX), wherein the transmitter (TX) is configured to emit high-frequency radiation when in operation, wherein the first transmitting antenna (ANT_TX1) and the second transmitting antenna (ANT_TX2) are configured to emit high-frequency radiation during operation so that radiation can reach the container (B), wherein first receiving antenna (ANT_RX1) is configured to record high-frequency radiation reflected from the container (B), wherein the receiver (RX) is configured to take up the high-frequency radiation received by the receiving antenna (ANT_RX1), wherein the control unit (C) is configured to control the transmitters so that the transmitter (TX) emits high-frequency radiation, and wherein the control unit (C) is also configured to evaluate high-frequency radiation taken up by the receiver (RX) so that a measurement of the volume of the liquid in the container (B) is determined, wherein the measurement of the volume of liquid in the container (B) is determined from channel state information. The invention also relates to device (1) for measuring volumes of a liquid in a container (B) by means of measuring emitted high-frequency radiation, comprising a control unit (C), a transmitter (TX), at least one first transmitting antenna (ANT_TX1) and at least one second transmitting antenna (ANT_TX2), a least one first receiving antenna (ANT_RX1) and a second receiving antenna (ANT_RX2) and a receiver (RX), wherein the transmitter (TX) is configured to emit high-frequency radiation when in operation, wherein the first transmitting antenna (ANT_TX1) and the second transmitting antenna (ANT_TX2) are configured to emit high-frequency radiation during operation so that radiation can reach the container (B), wherein the first receiving antenna (ANT_RX1) is configured to record high-frequency radiation reflected from the container (B), wherein the second receiving antenna (ANT_RX2) is configured to record high-frequency radiation transmitted from the container (B), wherein the control unit (C) is configured to control the transmitters so that the transmitter (TX) emits high-frequency radiation, and wherein the control unit (C) is also configured up to evaluate high-frequency radiation taken up by the receiver (RX) so that a measurement of the volume of the liquid in the container (B) is determined, wherein the measurement of the volume of liquid in the container (B) is determined from channel state information.

The invention relates to a device for measuring volumes of a liquid in a container by means of emitted high-frequency radiation.

Measuring volumes of a liquid by means of a measuring container is known. However, the transferring of liquids into a measuring vessel is not always practicable. For example, there are liquids which outgas during transferring or in the case of which part of the material to be measured evaporates. Other liquids may react with the ambient gases. For hygienic reasons, yet other liquids should come into contact with other materials as little as possible.

Determining volumes at a known density by means of measuring the weight is also known. However, in such measurements the weight of the container holding the liquid must then also be known. If this is not known beforehand, volume determination can only take place after emptying out the liquid or as a differential measurement. This is often disadvantageous. Furthermore, weighing devices are comparatively expensive and complex in design.

### OBJECTIVE

On the basis of this, one objective of the invention is to provide a simple and/or cost-effective possibility of determining liquids in containers, in particular in flexible containers. Preferably measurement should be able to take place promptly, more particularly in real time.

### BRIEF DESCRIPTION OF THE INVENTION

The objective is achieved by a device for measuring volumes of a liquid in a container by means of measuring emitted high-frequency radiation, comprising a control unit, a transmitter, at least one first transmitting antenna and at least one second transmitting antenna, at least one first receiving antenna and a receiver, wherein the transmitter is configured to emit high-frequency during operation, wherein the first transmitting antenna and the second transmitting antenna are configured to emit the high-frequency radiation during operation so that radiation can reach the container, wherein the receiving antenna is configured to receive the high-frequency radiation reflected by the containers, wherein the receiver is configured to take up the high-frequency radiation received by the receiving antenna, wherein the control unit is configured to control the transmitter in such a way that the transmitter emits high-frequency radiation and wherein the control unit is also configured to evaluate the high-frequency radiation received by the receiver in such a way that a measurement for the volume of the liquid in the container is determined, wherein the measurement of the volume of liquid in the container is determined from channel state information.

The objective is also achieved by a device for measuring volumes of a liquid in a container by means of measuring emitted high-frequency radiation, comprising a control unit, a transmitter, at least one first transmitting antenna and at least one second transmitting antenna, at least one first receiving antenna and a receiver, wherein the transmitter is configured to emit high-frequency during operation, wherein the first transmitting antenna and the second transmitting antenna are configured to emit the high-frequency radiation during operation so that radiation can reach the container, wherein the receiving antenna is configured to record high-frequency radiation reflected by the containers, wherein the receiver is configured to record the high-frequency radiation recorded by the receiving antenna, wherein the control unit is configured to control the transmitter in such a way that the transmitter emits high-frequency radiation and wherein the control unit is also configured to evaluate the high-frequency radiation recorded by the receiver on the basis of received digital data packages in such a way that a measurement for the volume of the liquid in the container is determined, wherein the measurement for the volume of liquid in the container is determined from channel state information.

Further advantageous embodiments are the subject matter of each of the dependent claims, the figures and the description.

### BRIEF DESCRIPTION OF THE FIGURES

The invention will be described in more detail below with reference to the figures. In these:

FIG. 1 shows a schematic view of elements in forms of embodiment of the invention,

FIG. 2 shows a schematic arrangement of antennae in relation to container according to forms of embodiment of the invention,

FIG. 3 shows a schematic arrangement of antennae in relation to container according to alternative or additional aspects in forms of embodiment of the invention,

FIG. 4 shows a schematic arrangement of antennae in relation to container according to alternative or additional aspects in forms of embodiment of the invention, and

FIG. 5 shows a schematic arrangement of antennae in relation to container according to alternative or additional aspects in forms of embodiment of the invention.

### DETAILED DESCRIPTION OF THE INVENTION

The invention will be described in more detail below with reference to the figures. It should be noted that different aspects are described which can each be used individually or in combination. This means that each aspect can be used with different forms of embodiment of the invention unless explicitly set out as a pure alternative.

Furthermore, for the sake of simplicity reference is generally only made to one entity. However, unless specifically stated, the invention can also comprise several of the entities in question. Thus, the use of the words “a” and “an” should only be taken as an indication that in a simple form of embodiment at least one entity is used.

Insofar as methods are described below, the individual steps of a method can be arranged and/or combined in any order unless otherwise explicitly evident from the context. In addition, the methods—unless expressly characterised differently—can be combined with each other.

As a rule, indicated numerical values should not be understood as exact values, but contain a tolerance of +/−1% to +/−10%.

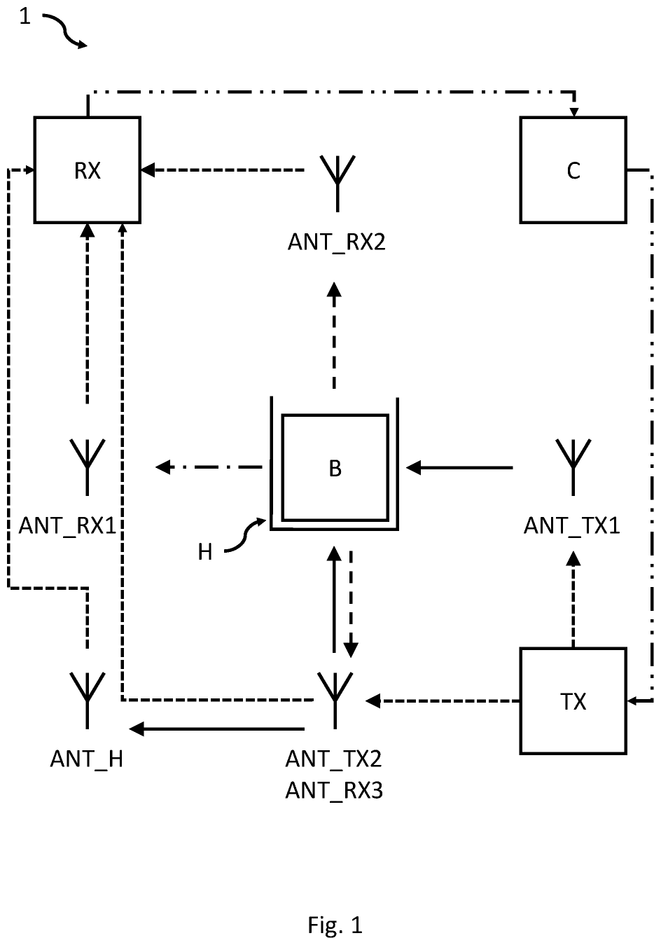

Below we will refer in particular to FIG. 1 in which a schematic overview of elements in forms of embodiment of the invention is shown. This means that not all elements are necessary for the solution according to the invention.

In a first form of embodiment of the invention a device 1 for measuring volumes of a liquid in a container B by means of measuring emitted high-frequency radiation is provided.

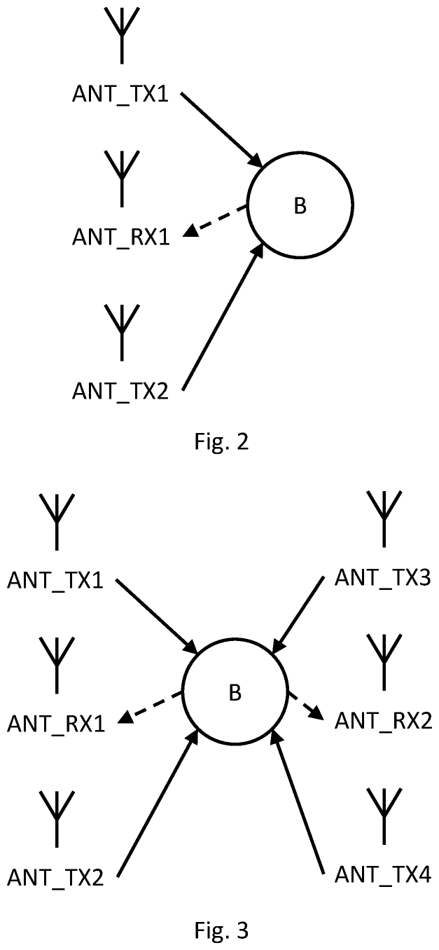

The device 1 comprises a control unit C, a transmitter TX, at least one first transmitting antenna ANT_TX1 and at least one second transmitting antenna ANT_TX2, at least one first receiving antenna ANT_RX1 and a receiver RX. Such an arrangement is schematically shown in FIG. 2.

The transmitter TX is configured to emit high-frequency radiation when in operation. The radiation can be modulated to one or more frequencies. The high-frequency radiation carries digital data packages.

The first transmitting antenna ANT_TX1 and the second transmitting antenna ANT_TX2 are configured to emit the high-frequency radiation during operation so that radiation can reach the container B.

In turn, in operation the receiving antenna ANT_RX1 is configured to receive the high-frequency radiation reflected from the container B.

In other words, the device 1 comprises a predetermined arrangement of transmitter antenna(e), container B and receiving antenna(e).

In operation, the receiver RX is configured to take up the high-frequency radiation received by the receiver antenna ANT_RX1.

The control unit C is configured to control the transmitter TX in such a way that the transmitter TX emits high-frequency radiation. In other words, through being controlled the transmitter TX is induced to emit high-frequency radiation (via one or more antennae) (at one or more frequencies) in controlled manner.

The control unit C is also configured to evaluate the high-frequency radiation taken up by the receiver RX (via one or more antennae) (at one or more frequencies) on the basis of received digital data packages so that a measure of the volume of the liquid in the container B is determined.

Preferably the measurement of the volume of liquid in the container B is determined from channel state information.

Channel state information is used in many wireless (digital) communication systems to characterise the properties of a communication channel. The channel state information thus reflects properties along the propagation path which, for example, are influenced by dispersion, attenuation, drop in performance due to distance etc.

By evaluating channel state information, pointers, for example, can obtained as to how transmitting properties should be changed so that in the case of given channel properties a reliable connection with preselected properties (e.g. achieving a certain data rate) can be realised. However, in the invention it is not a question of this adaptability. For the invention, only the description of the propagation path is of interest. To this extent, other information, which reflect the properties of the propagation path in a similar way, can be used in the same way. The invention utilises the change of channel state information data packages in the propagation of the signal, in particular on passing through liquids: Certain packages exhibit errors after passing through a liquid. Knowledge of error emergence along the signal propagation is used to determine the liquid volume.

Without loss of generality, the device as in FIG. 2 can be arranged in such a way that the connection lines between the used transmitter antennae ANT_TX1 and ANT_TX2 in relation to the container B form an angle of 1° to 180°, preferably 30° to 90°.

In a second form of embodiment of the invention a device 1 for measuring volumes of a liquid in a container B by means of measuring emitted high-frequency radiation is provided.

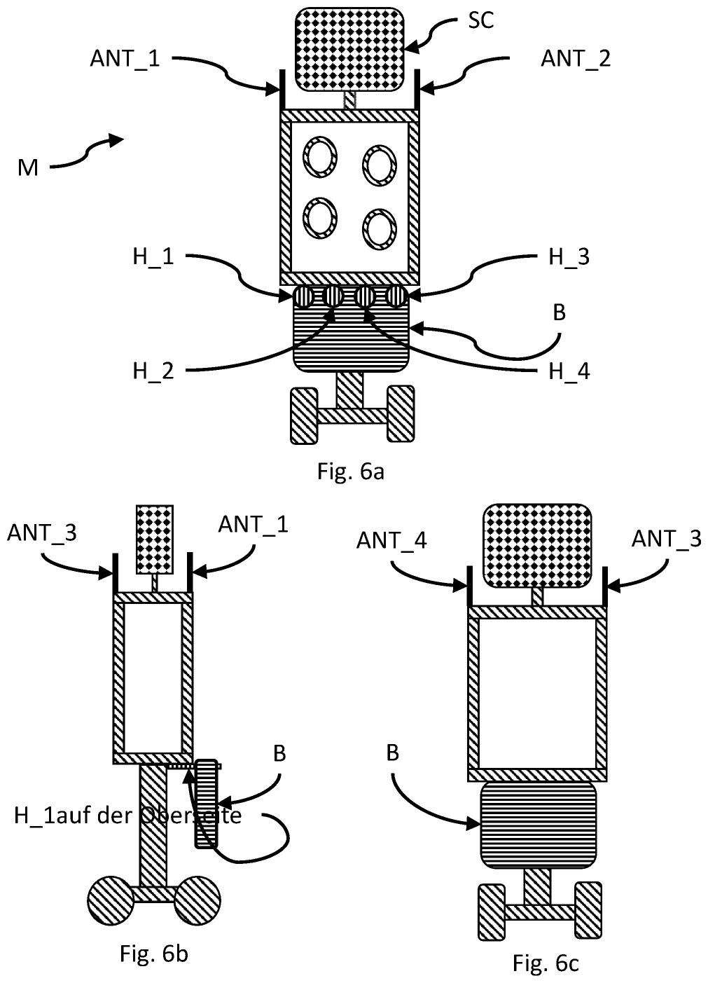

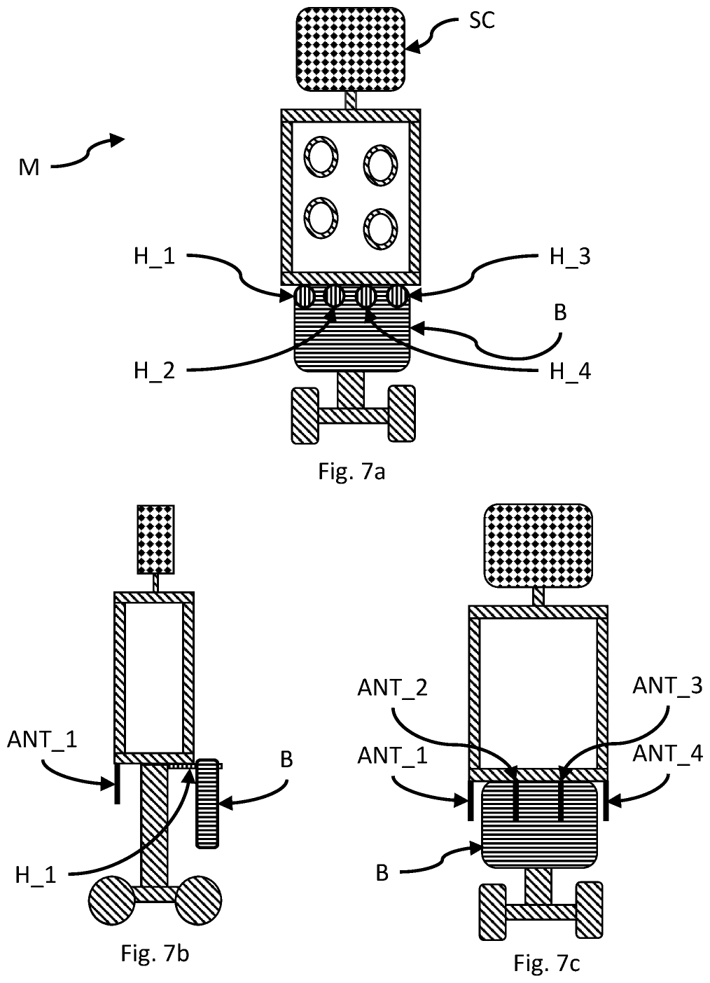

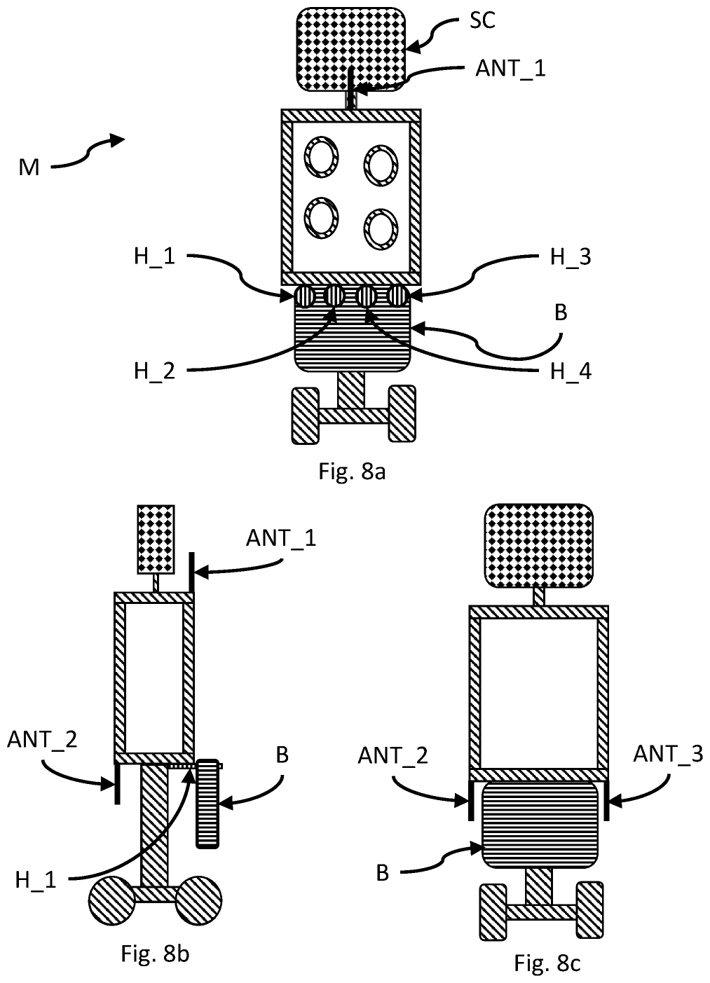

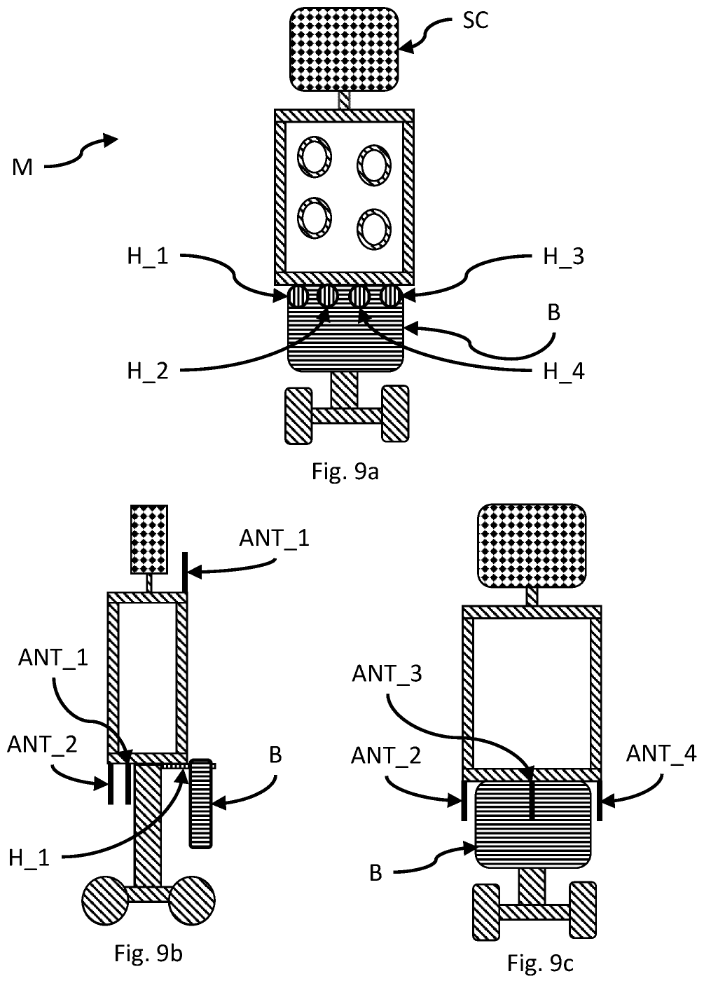

In turn, the device 1 comprises a control unit C, a transmitter TX, at least one first transmitting antenna ANT_TX1 and at least one second transmitting antenna ANT_TX2, at least one first receiving antenna ANT_RX1 and a second receiving antenna ANT_RX2 and a receiver RX. Such an arrangement is schematically shown in FIG. 5.

The transmitter TX is configured to emit high-frequency radiation when in operation.

The transmitter TX is configured to emit high-frequency radiation when in operation. The radiation can be modulated to one or more frequencies. The high-frequency radiation carries digital data packages.

The first transmitting antenna ANT_TX1 and the second transmitting antenna ANT_TX2 are configured to emit the high-frequency radiation during operation so that radiation can reach the container B.

When in operation, the first receiving antenna ANT_RX1 is configured to receive high-frequency radiation reflected from the container B.

On the other hand, the second receiving antenna ANT_RX2 is set up to take up high-frequency radiation transmitted from the container B.

In other words, the device 1 comprises a predetermined arrangement of transmitter antenna(e), container B and receiving antenna(e).

The control unit C is configured to control the transmitter in such a way that the transmitter TX emits high-frequency radiation. In other words, through being controlled the transmitter TX is induced to emit high-frequency radiation (via one of more antennae) (at one or more frequencies) in a controlled manner.

The control unit C is also configured to evaluate the high-frequency radiation take up by the receiver RX on the basis of received digital data packages so that a measurement of the volume of the liquid in the container B is determined.

Preferably the measurement of the volume of liquid in the container B is determined from channel state information.

Channel state information is used in many wireless communication systems to characterise the properties of a communication channel. The channel state information thus reflects properties along the propagation path which, for example, are influenced by dispersion, attenuation, drop in performance due to distance etc. The channel state information must be distinguished from the less informative RSSI (Received Signal Strength Indicator).

By evaluating channel state information, pointers, for example, can obtained as to how transmitting properties should be changed so that in the case of given channel properties a reliable connection with preselected properties (e.g. achieving a The invention utilises the change of channel state information data packages in the propagation of the signal, in particular on passing through liquids: Certain packages exhibit errors after passing through a liquid. Knowledge of error emergence along the signal propagation is used to determine the liquid volume. certain data rate) can be realised. However, in the invention it is not a question of this adaptability. For the invention, only the description of the propagation path property is of interest. To this extent, other information, which reflect the properties of the propagation path in a similar way, can be used in the same way.

This second form of embodiment of is particularly good for the recognition of liquids in bags, which in the case of volume changes tend to change in shape, e.g. through lateral displacement, buckling etc. When the volume of a liquid changes in flexible bag, wrinkling, buckling, displacement etc. can occur, which can have a disruptive effect on other measuring devices as it can lead to migration of a wall of the containers (namely the bag) relative to measuring devices such as sensor or antennae.

Although the devices 1 have been described separately above, it can be envisaged that both forms of embodiment are provided in a joint device. Thus, within a device 1, based on different measuring protocols, a measurement of the volume of the liquid in the container B could be determined from one piece of channel state information both simultaneously or also offset in time. Both thus determined measurements can then, for example, can be made available for plausibility testing and/or notification. It should be noted that through a suitable selection, one or more antennae can serve as transmitting or receiving antennae (e.g. for different spatial measurements in one form of embodiment or in a first measurement according to the first form of embodiment and in a second measurement according to to the second form of embodiment).

In other words, on the basis of a predetermined structure, in the case of all forms of embodiment the volume in a container B can be easily measured in a contactless manner.

Without restricting the generality of the invention, conventional hardware as found, in for example, WLAN devices can be used for this. Through this particularly cost-effective devices 1 can be made available. For example transmitter RX and transmitter TX and/or the assigned antennae can be components of a WLAN device. It is known that, for example, certain network chip sets make it possible to determine channel state information or make available the data forming the basis of this determination. An example of a chip set is marketed as the Atheros chip set. Chip sets which make this information available are generally also found in access points, such as, for example, WLAN-capable routers and MIMO-capable devices. A channel state information-capable chip set or a WLAN card is also offered by Intel for example.

Thus, with a single computer as control unit C and two network interfaces allowing the determination of a CTI value, a device 1 of this type can be realised.

In forms of embodiment according to the invention it is optionally envisaged that the distance between the first transmitting antenna ANT_TX1 and the first receiving antenna ANT_RX1 is at least ⅜ of the used wavelength of the high-frequency radiation to be emitted.

In forms of embodiment according to the invention it is also optionally envisaged that the distance between the first transmitting antenna ANT_TX1 and the first receiving antenna ANT_RX1 in relation to the container B is at least ⅜ of the used wavelength of the high-frequency radiation to be emitted.

In forms of embodiment according to the invention it is optionally envisaged that the distance between the first transmitting antenna ANT_TX1 and the first receiving antenna ANT_RX1 is around 4 times the used wavelength of the high-frequency radiation to be emitted.

In embodiments of the invention it is also envisaged that the high-frequency radiation is selected from radiation of a near-field communication system or radiation of a frequency permitted for use for industrial, scientific, medical, domestic or similar purpose that is not a radiocommunication application.

Typical near-field communications systems are, for example, WLAN, Bluetooth (low energy), Zigbee, DECT (ultra low energy) or their successor systems, without being restricted to a particular specification. Typical frequencies which are approved for industrial, scientific, medical, domestic or similar purposes which are not a radio application are to be found the frequency ranges 433.05 MHz-434.79 MHz, 902 MHz-928 MHz, 2.4 GHz-2.5 GHz, 5.725 GHz-5.875 GHz, 24 GHz-24.25 GHz, 61 GHz-61.5 GHz, 122 GHz-123 GHz as well as 244 GHz-246 GHz, but without being limited thereto.

However, in one form of embodiment of the invention high-frequency radiation with a frequency in the range 2 GHz to 4 GHz, more particularly 2.4 GHz and more particularly signals in the WLan spectrum and/or in accordance with WLAN specification IEEE 802.11 IEE 802.11b IEEE 802.11g IEEE 802.11n according to the summary in IEE 002-11-2012 are used.

In a further form of embodiment of the invention the container B is a bag. Bags are characterised in that these are generally closed and via a controlled opening the liquid can flow into the bag(s). Moreover, bags can change their external shape, e.g. if liquid is removed from the container B. In particular this means that if a bag B provides a greater volume than the liquid in the bag B requires, the outer shape will be able to change under the effect of gravity, for example.

Bags as container B represent a great challenge for volume determination, but are easy to manage within the framework of the invention.

In one form of embodiment of the invention at least one transmitting antenna ANT_TX1 is applied to the container B or a receptacle H. For example, an antenna can be printed on or stuck on. By means of a suitable contact device the antenna can then be brought into contact with the transmitter. Provision of an antenna on the container B or receptacle H can, for example, be advantageous if the distance between the transmitting antenna and the container B or the liquid is to be small or defined.

In a further form of embodiment of the invention at least one receiving antenna ANT_RX1 is applied to the container B or a receptacle H. For example, an antenna can be printed on or stuck on. By means of a suitable contact device the antenna can then be brought into contact with the transmitter. Provision of an antenna on the container B or receptacle H can, for example, be advantageous if the distance between the receiving antenna and the container B or the liquid is to be small or defined.

The place of attachment of such a transmitting antenna or receiving antenna can, for example, be selected by way of properties of the container B, so that, for example, the radiation can pass through the liquid as independently as possible from the filling level of the liquid in the container B. A transmitting antenna or a receiving antenna can be arranged on the base of the container B for example.

In one form of embodiment of the invention the container B has a flexible wall. Then it can be envisaged that the device 1 for measuring—as shown in FIG. 1—comprises a receptacle H with a rigid wall so that the container B in a filled state adjoins the receptacle H laterally.

For example, the wall can be so high that a bag B completely filled with liquid when present in the receptacle H does not protrude beyond the wall. For example, the receptacle H can be in the form of a rigid container, for instance a bath or drawer. It can, for example, be made of plastic.

The surface area of the receptacle H can, for example, be selected to be such that a bag B completely filled with a liquid can be introduced into the receptable H. The surface area of the receptacle H can be selected to be such that a bag B completely filled with a liquid is in contact with the wall on around 50% of the wall surface of the bag.

The surface area can of course also be determined by other considerations. It can, for example, be desirable that the basic dimensions of the surface area, e.g. the diameter, do not fall below a certain size, e.g. at least one wavelength of the radiation used.

In one form of embodiment the receptable H is in the form of one or more mandrels or rods, on which a bag can be suspended. Here, the bag can, for example, have eyelets so that on suspension mandrels or rods project through corresponding eyelets.

In a further form of embodiment of the invention the device 1 also comprises a receiving antenna ANT_H for determining background radiation. The background radiation can also be determined by means of one or more already present receiving antennae. This possible, for example, at times when the receiving antenna is not required for other types of measurement.

With auxiliary antennae, in particular aligned auxiliary antennae (possible both as receiving and transmitting antennae), the proportion of attenuation through free space emission can be determined very precisely, for example, through which a correcting parameter can be determined. If the influence of free space attenuation is small, this determination can be dispensed with.

Without restricting the generality of the invention, a transmitting antenna (or several or all) ANT_TX1, ANT_TX2 can have a directional characteristic alternatively to an omnidirectional characteristic.

Without restricting the generality of the invention, a receiving antenna (or several or all) ANT_RX1, ANT_RX2, ANT_RX3, ANT H can have a directional characteristic alternatively to an omnidirectional characteristic.

Omnidirectional characteristics are provided by a rod antenna for example. Directional characteristics are shown by dipole-type antennae or panel antennae for example.

The invention can be used in many sectors.

However, it is of particular importance in the field of medicine. In the field of medicine there are numerous medical devices M in which a weight or a volume of a liquid, are monitored, during a treatment for example.

For example, a medical device M can measure the volume of a liquid in a container B to be supplied to the body of a mammal or removed from the body of a mammal, or a liquid in a secondary circulation for treating this liquid. Examples of liquid supplied to the body of a mammal are, for example, infusions, heparin, blood, saline solutions, drugs for intravenous administration, parenteral feeding etc. Examples of liquids removed from the body of a mammal are blood, urine.

In particular the medical device M can be a dialysis device, wherein the liquid is a liquid in connection with dialysis, more particularly dialysate. The form of dialysis is not fixed, but can, for example, be in the form of renal dialysis in the form of haemodialysis, peritoneal dialysis, haemofiltration, haemodiafiltration and haemoperfusion, or also relate to hepatic dialysis, in particular apheresis, single-pass albumin dialysis, molecular adsorbents recirculation system.

Preferably the medical device M is a dialysis machine and the dialysis measures the volume of a liquid in one or more bags. In a preferred embodiment the dialysis machine is connected to a bag B for fresh dialysate and/or for used dialysate. The dialysis machine can determine the liquid balance during a treatment through the measurement of fresh and used dialysate. In a further embodiment a dialysis machine M has one or more receptacle(s), e.g. for suspending one or more containers B, e.g. bags, e.g. for dialysate, on an lower edge, and a device 1 according to the invention for measuring the volume of a liquid, in such a way that by means of high-frequency radiation the dialysis machine M can determine the liquid volume in the suspended containers B.

Here the antennae ANT_1 . . . ANT_5 . . . ANT_N of the device can be arranged accordingly. FIGS. 6 to 9 schematically show different points of application in relation to a medical device M. The medical device M comprises, for example, an optional display SC, e.g. a (flat) screen) on which the results relating to one or more volumetric measurements, e.g. current volume, volume change, volumetric flow etc. can be shown. However, at the same time the optional display SC can also provide a user interface with which, for example, measurement by the device 1 can be manually brought about. In figure several receptacles H_1, H_2, H_3 H_4 are shown. However, only one receptable H or more receptacles can be provided. Equally, instead of one container B, several containers can be provided.

The antennae ANT_1 . . . ANT_4 . . . ANT_N of the device 1 can, for example, as shown in FIGS. 6a-6c, be arranged on the upper side of the medical device. However, as shown in FIGS. 7a-7c the antennae can also be arranged on the underside of the medical device M. However, other arrangements are not ruled out out by this. For example, as shown in FIGS. 8a-8c the antennae can also be arranged in a distributed manner. Whereas ANT_1 tends to be arranged centrally on the front side, antennae ANT_2 and ANT_3 are arranged distributed on the rear side. In FIGS. 9a-9c antenna ANT_1, for example, is offset with regard to antennae ANT_2 . . . ANT_4.

The function of the antennae ANT_1 . . . ANT_5 . . . ANT_N of the device 1, i.e. as transmitting antenna and/or as receiving antenna can be suitably selected.

Through this, for example, expensive and laborious scales can be spared and there is the advantage that heavy bags B only have to be suspended on at the bottom of the housing of the medical device M and do not have to be placed on a scales dish.

In this way handling is made easier. Such medical devices M can be used in regions with an unsteady water supply, in cases of temporary or mobile deployment or in intensive care wards.

For example, the medical device in FIGS. 6-9 can be a dialysis treatment machine (in particular a haemodialysis machine) with a device 1 according to the invention. In such dialysis treatment machines the filling level, for example, is measured (and monitored) in a connected container B. The container B is typically a 5 L plastic canister. A typical liquid stored in such a container B is a concentrate for dialysis treatment. The liquids contain acetates or bicarbonates for example.

In this way it can particularly advantageously be achieved that the container(s) B are not emptied unexpectedly during a treatment and the desired treatment parameters cannot be observed or air is drawn in by the pump etc.

In all forms of embodiment it is envisaged that the measurement for the volume of the liquid in the container B is determined via a plurality of individual measurements, e.g. several 10 of thousands of measurements, for example 27 thousand measurements. In doing so a plurality of data packages are sent and received. The accompanying parameters, such as the channel state information itself is a mean value or can be determined itself.

In addition, in all forms of embodiment it can be envisaged that the measuring arrangement of transmitting antennae and receiving antenna is multiply present.

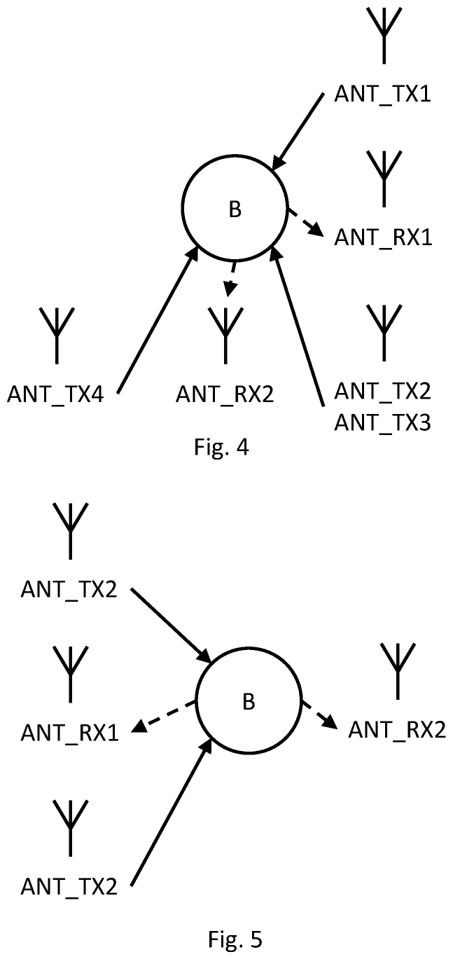

If a device according to FIG. 2 is multiply provided, it can, for example be envisaged that the arrangements are at an angle of 15° to 135° with regard to each other, as shown in FIG. 4.

In FIG. 3 a first arrangement could comprise the transmitting antennae ANT_TX1, ANT_TX2 and the receiving antennae ANT_RX1, wherein as a mirror image thereto a second arrangement comprises the transmitting antennas ANT_TX3, ANT_TX4 and the receiving antenna ANT_RX2.

The arrangements could have very generally different positions with regard to each other and/or the device could be built up differently with regard to each other.

1. A device for measuring volumes of a liquid in a container by means of measuring emitted high-frequency radiation, comprising
a control unit,
a transmitter, at least one first transmitting antenna and at least one second transmitting antenna, at least one receiving antenna and a receiver,
wherein the transmitter is configured to emit high-frequency radiation when in operation, wherein the first transmitting antenna and the second transmitting antenna are configured to emit high-frequency radiation during operation so that radiation can reach the container,
wherein first receiving antenna is set up to record high-frequency radiation reflected from the container,
wherein the receiver is set up to take up the high-frequency radiation received by the receiving antenna,
wherein the control unit is configured to control the transmitters so that the transmitter emits high-frequency radiation, and wherein the control unit is also set up to evaluate high-frequency radiation taken up by the receiver so that a measure of the volume of the liquid in the container is determined,
wherein the measurement of the volume of liquid in the container is determined from channel state information.

2. A device for measuring volumes of a liquid in a container by means of measuring emitted high-frequency radiation, comprising
a control unit,
a transmitter, at least one first transmitting antenna and at least one second transmitting antenna, a least one first receiving antenna and a second receiving antenna and a receiver,
wherein the transmitter is configured to emit high-frequency radiation when in operation, wherein the first transmitting antenna and the second transmitting antenna are configured to emit high-frequency radiation during operation so that radiation can reach the container,
wherein the first receiving antenna is configured to record high-frequency radiation reflected from the container,
wherein the second receiving antenna is configured to record high-frequency radiation transmitted from the container,
wherein the control unit is configured to control the transmitters so that the transmitter emits high-frequency radiation, and wherein the control unit is also configured up to evaluate high-frequency radiation taken up by the receiver so that a measurement of the volume of the liquid in the container is determined,
wherein the measurement of the volume of liquid in the container is determined from channel state information.

3. The device according to claim 1, wherein the distance between the first transmitting antenna and the first receiving antenna s at least ⅜ of the used wavelength of the high-frequency radiation to be emitted.

4. The device according to claim 1, wherein the distance between the first transmitting antenna and the first receiving antenna is around 4 times the used wavelength of the high-frequency to be emitted.

5. The device according to claim 1, wherein the high-frequency radiation is selected from radiation of a near-field communication system or radiation of a frequency permitted for use for industrial, scientific, medical, domestic or other purpose that is not a radiocommunication application.

6. The device according to claim 1, wherein the high-frequency radiation comprises radiation of a frequency in the range 2 GHz to 4 GHz.

7. The device according to claim 1, wherein the container is a bag.

8. The device according to claim 1, wherein the transmitting antenna is applied on the container or a receptacle.

9. The device according to claim 1, wherein the receiving antennais applied on the container or a receptacle.

10. The device according to claim 1, wherein the container comprises a flexible wall, wherein the device for measuring a receptacle has a rigid wall so that the container in a filled state is laterally in contact with the receptacle.

11. The device according to claim 1, wherein the device also comprises a receiving antenna for determining background radiation.

12. The device according to claim 1, wherein the at least one transmitting antenna has a directional characteristic.

13. The device according to claim 1, wherein the at least one receiving antenna has a directional characteristic.

14. A medical device comprising a device according to claim 1.

15. The medical device according to claim 14, wherein the liquid, the volume of which is to be measured in the container, is supplied to the body of a mammal or removed therefrom, or is a liquid in a secondary circulation for treating this liquid.

16. The medical deviceaccording to claim 14, wherein the medical device is a dialysis device and wherein the liquid is a liquid in connection with dialysis.
